(19) United States
(12) Patent Application Publication
(10) Pub. No.: US 2023/0170040 A1
(43) Pub. Date: Jun. 1, 2023

(54) PRIORITIZING POTENTIAL NODES FOR EDITING OR POTENTIAL EDITS TO A NODE FOR STRAIN ENGINEERING

(71) Applicant: Zymergen Inc., Emeryville, CA (US)

(72) Inventor: Trent Hauck, Seattle, WA (US)

(73) Assignee: Zymergen Inc., Emeryville, CA (US)

(21) Appl. No.: 17/420,629

(22) PCT Filed: Jan. 3, 2020
(86) PCT No.: PCT/US2020/012252

§ 371 Date: Jul. 2, 2021

Related U.S. Application Data
(60) Provisional application No. 62/789,449, filed on Jan. 7, 2019.

(51) Int. Cl. G16B 5/20; G16B 40/20; G16B 45/00

(52) U.S. Cl. CPC G16B 5/20; G16B 40/20; G16B 45/00

(57) ABSTRACT

A microorganism engineering project employs a vector representation of a biological network. The vector representation allows researchers to input elements known to have a direct impact on a goal of the project (e.g., an element in the metabolic pathway for generating a compound of interest). Within the vector representation, other biological elements are positioned with respect to an input element based on biological relatedness. In this manner, based on positions within the vector representation, candidate elements may be identified for editing. For example, if the elements are associated with a gene, the gene sequence may be knocked out and/or a different promoter may be used for regulating the gene.

### CROSS-REFERENCE TO RELATED APPLICATIONS

This application is a 371 US National Phase of International application no. PCT/US2020/012252, filed Jan. 3, 2020, which claims the benefit of U.S. provisional application No. 62/789,449, filed Jan. 7, 2019, each of which is hereby incorporated by reference in its entirety.

### FIELD

This disclosure pertains to computational systems and methods for modeling biological systems.

### BACKGROUND

Genome engineering may be implemented in many ways. Many approaches target multiple loci in a strain's genome. Starting from a candidate strain that may be wildtype or may already have a set of genetic changes compared to the wildtype, the design decision involves selection of one or more target loci and selection of the perturbation strategy. Perturbations to a locus are sometimes referred to as “edits.”

When a locus is selected for genetic perturbation, different types of changes can be applied to the locus. The change types might be anything from a single nucleotide change, to a promoter swap, to the insertion or deletion of a gene.

Due to the size of microbial genomes, many genes might not have been previously targeted, but prior biological information about such genes is often available from various public and private sources. Further information might be available about complicating characteristics such as reaction types carried out by various genes, growth conditions, etc. It would be desirable to have an accurate predictive modeling method that is capable of incorporating large amounts of information including, for example, prior biological knowledge.

### SUMMARY

One aspect of the disclosure provides methods of identifying one or more elements for modification to cause a change in functioning of a microorganism. Such methods may be characterized by the following operations: (a) receiving a graph representation of a biological network of interacting elements of a microorganism or a plurality of related microorganisms; (b) converting the graph representation of the biological network to a vector representation having locations of the interacting elements, which locations are derived from positions of said interacting elements in the graph representation; (c) determining distance relationships between the interacting elements as represented in the vector representation; and (d) from the distance relationships determined in (c), recommending a subset of the interacting elements for modification in an engineered variant of the microorganism or the plurality of related microorganisms.

In certain embodiments, recommending a subset of the interacting elements for modification comprises generating sampling probabilities for selecting at least some of the interacting elements for modification.

In certain embodiments, a method additionally includes shifting the sampling probabilities to become more sparse. In such embodiments, shifting the probabilities to become more sparse may employ hyperparameters when generating sampling probabilities for at least some of the interacting elements.

In certain embodiments, a method additionally includes modifying a first one of the interacting elements in the subset of interacting elements recommended in (d) to produce the engineered variant microorganism. In some such embodiments, the first one of the interacting elements is a gene. In some cases, modifying the first one of the interacting elements comprises mutating the gene or knocking out the gene.

In some methods, the graph representation of the biological network is a graph representation of a metabolic network of the microorganism or the plurality of related microorganisms. In some methods, the graph representation of the biological network is an N-partite graph comprising genes, compounds, and reactions in the microorganism or the plurality of related microorganisms. In certain embodiments, the graph representation of the biological network includes two or more interacting elements selected from: genes, compounds, reactions, genetic ontology terms, transcriptional units, and proteins.

In certain embodiments, the graph representation of the biological network includes: a first edge representing a first compound that is a reactant for a first metabolic reaction, a second edge representing a second compound that is a product of the first metabolic reaction, and a third edge representing a first gene facilitating the first metabolic reaction.

In certain embodiments, the graph representation of the biological network includes two or more edges selected from the group consisting of a first edge representing a first compound that is a reactant for a first metabolic reaction, a second edge representing a second compound that is a product of the first metabolic reaction, a third edge representing a first gene facilitating the first metabolic reaction, a fourth edge representing a first protein encoded by the first gene, and a fifth edge representing a genetic ontology term for the first protein.

The operation of converting the graph representation of the biological network to a vector representation may include: (i) conducting a plurality of random walks through the graph representation of the biological network and monitoring the interacting elements encountered during the random walks; and (ii) using information about the random walks to define positions of interacting elements in the vector representation. In some cases, the information about the random walks comprises frequencies at which the interacting elements of the graph representation were encountered during the random walks.

In certain embodiments, converting the graph representation of the biological network to a vector representation includes employing information about a hierarchy of the interacting elements of the biological network. In some such embodiments, converting the graph representation of the biological network to a vector representation includes presenting the hierarchy of the interacting elements of the biological network in a hyperbolic space. Further, presenting the hierarchy of the interacting elements of the biological network in a hyperbolic space may include performing a hyperbolic embedding. As an example, the hyperbolic embedding is a Poincare embedding.

Some methods additionally include identifying an input element from among the interacting elements. In such methods, determining distance relationships between the interacting elements in (c) may include: (i) determining a first distance relationship between the input element and a first one of the other interacting elements, and (ii) determining a second distance relationship between the input element and a second one of the other interacting elements.

In certain embodiments, the input element participates in a reaction that produces a specified result in the microorganism or the plurality of related microorganisms. In some cases, the input element is a protein, a gene encoding the protein, a genetic ontology feature for the protein, an intermediate compound that interacts with the protein, or reaction pathway containing the protein. In some cases, the specified result is a production of specified compound by a reaction involving the protein.

In certain embodiments, the methods additionally include producing the engineered variant of the microorganism or the plurality of related microorganisms; and testing the engineered variant of the microorganism or the plurality of related microorganisms. In certain embodiments, the methods additionally include: (i) growing the engineered variant in a reactor; and (ii) producing a product of the engineered variant in the reactor.

Some aspects of the disclosure may involve methods of identifying promoter-gene combinations for editing in a microorganism genome. Such methods may be characterized by the following operations: (a) receiving a graph representation of a biological network of interacting elements of a microorganism or a plurality of related microorganisms, wherein the interacting elements comprise genes present in the microorganism or microorganisms; (b) converting the graph representation of the biological network to a vector representation having locations of the interacting elements, which locations are derived from positions of said interacting elements in the graph representation; (c) determining distance relationships between the interacting elements, including at least some of the genes present in the microorganism or microorganisms, as represented in the vector representation; and (d) from the distance relationships determined in (c), ascribing probabilities of promoter-gene combinations for use in an engineered variant of the microorganism or the plurality of related microorganisms.

In certain embodiments, ascribing probabilities of promoter-gene combinations includes using the distance relationships to generate sampling probabilities for selecting a subset of the genes present in the microorganism or microorganisms. In certain embodiments, ascribing probabilities of promoter-gene combinations includes using angles between interacting elements in the vector representation to suggest a first promoter from among a plurality of promoters for use with a first one of the genes present in the microorganism or microorganisms.

In certain embodiments, using angles between interacting elements in the vector representation includes determining cosine similarities between interacting elements in the vector representation.

Some methods additionally include shifting the probabilities ascribed in (d) to become more sparse. As an example, shifting the probabilities ascribed in (d) to become more sparse includes employing hyperparameters when ascribing probabilities of promoter-gene combinations.

Various other features of these methods correspond to those additional features presented above for the first method aspect of the disclosure.

Some aspects of the disclosure pertain to systems for creating a variant microorganism for producing a product. Such systems may be characterized by the following features: (a) a computing device comprising one or more processors and memory, and (b) a genetic engineering tool configured to produce the variant microorganism. The computing device may be configured to: (i) receive a graph representation of a biological network of interacting elements of a microorganism or a plurality of related microorganisms, (ii) convert the graph representation of the biological network to a vector representation having locations of the interacting elements, which locations are derived from positions of said interacting elements in the graph representation, (iii) determine distance relationships between the interacting elements as represented in the vector representation, and (iv) from the distance relationships determined in (c), recommend a subset of the interacting elements for modification in an engineered variant of the microorganism or the plurality of related microorganisms.

In certain embodiments, the system additionally includes a bioreactor configured to produce the product from the modified organism. In certain embodiments, the genetic engineering tool configured to produce the modified organism is configured to apply a mutation to the gene or knock out the gene. In certain embodiments, the genetic engineering tool configured to produce the modified organism includes a gene editing tool such as a TALEN system, a zinc finger system, or a CRISPR/Cas9 system designed to apply the mutation to the gene.

In certain embodiments, the computing device is configured to recommend the subset of the interacting elements for modification by generating sampling probabilities for selecting at least some of the interacting elements for modification. As an example, the computing device may be further configured to shift the sampling probabilities to become more sparse.

In certain embodiments, the computing device is further configured to modify a first one of the interacting elements in the subset of interacting elements recommended in (iv) to produce the engineered variant microorganism. In some examples, when the first one of the interacting elements is a gene and when the system modifies the first one of the interacting elements, it mutates the gene or knocking out the gene.

In certain embodiments, the graph representation of the biological network is a graph representation of a metabolic network of the microorganism or the plurality of related microorganisms. In certain embodiments, the graph representation of the biological network is an N-partite graph comprising genes, compounds, and reactions in the microorganism or the plurality of related microorganisms. In certain embodiments, the graph representation of the biological network includes two or more interacting elements selected from the group consisting of genes, compounds, reactions, genetic ontology terms, transcriptional units, and proteins. In certain embodiments, the graph representation of the biological network includes: a first edge representing a first compound that is a reactant for a first metabolic reaction, a second edge representing a second compound that is a product of the first metabolic reaction, and a third edge representing a first gene facilitating the first metabolic reaction.

In certain embodiments, the graph representation of the biological network includes two or more edges selected from: a first edge representing a first compound that is a reactant for a first metabolic reaction, a second edge representing a second compound that is a product of the first metabolic reaction, a third edge representing a first gene facilitating the first metabolic reaction, a fourth edge representing a first protein encoded by the first gene, and a fifth edge representing a genetic ontology term for the first protein.

In certain embodiments, the computing device is configured to convert the graph representation of the biological network to a vector representation by:
conducting a plurality of random walks through the graph representation of the biological network and monitoring the interacting elements encountered during the random walks; and
using information about the random walks to define positions of interacting elements in the vector representation.

In certain embodiments, the information about the random walks includes frequencies at which the interacting elements of the graph representation were encountered during the random walks.

The computing device may be configured to convert the graph representation of the biological network to a vector representation by employing information about a hierarchy of the interacting elements of the biological network. In some such cases, the computing device is further configured to convert the graph representation of the biological network to a vector representation by presenting the hierarchy of the interacting elements of the biological network in a hyperbolic space. As an example, the hyperbolic space is a Poincare space.

In certain embodiments, the computing device is further configured to identify an input element from among the interacting elements. In such embodiments, the computing device may be configured to determine distance relationships between the interacting elements in (iii) by determining a first distance relationship between the input element and a first one of the other interacting elements, and determining a second distance relationship between the input element and a second one of the other interacting elements.

In general, the computing device may be configured to perform any one or more of the computational operations described in method aspects of the disclosure and/or control any or morer of the physical operations described in the method aspects.

Other aspects of the disclosure pertain to microorganisms including a modification to a gene or a gene-promoter combination where the modification is selected using any of the above described method aspects of the disclosure. In certain embodiments, the modification to the gene includes mutating the gene or knocking out the gene.

These and other features of the disclosure will be presented below with reference to the associated drawings.

### BRIEF DESCRIPTION OF THE DRAWINGS

FIG. 1 presents a simple schematic example of the graph and vector space relationship.

FIG. 2 illustrates an example process flow for conducting a first approach to generating a vector space.

FIG. 3 illustrates an example process flow for conducting a second approach to generating a vector space, typically one that is hyperbolic or non-Euclidean.

FIG. 4 illustrates an example process flow for using a vector space to identify elements of a biological system to edit in a strain engineering project.

FIG. 5 depicts an example process of creating sampling probabilities from a metabolite in a node embedding space.

FIG. 6 depicts an example process of creating sampling probabilities for promoter swaps using a node embedding space.

FIG. 7 presents an example of a computer architecture that may be employed to receive data representing a biological network such as a metabolic network for a microorganism, produce a vector representation of a biological network in which the biological relatedness of the nodes is represented by distances between nodes in the representation, use a vector representation to predict the impact of an edit to a node in the biological network, compare edits, optionally control operations associated with genome engineering (e.g., high throughput genome modifications, microorganism growth conditions, assaying), and the like.

### DETAILED DESCRIPTION

Overview and Context

A typical microorganism engineering project searches for better performing microorganism strains. Such project might identify one or more genes or other elements that are relevant to the project and, as such, are candidates for modifying (sometimes called editing). For example, the project might involve engineering a microorganism to increase its output of a particular compound. The relevant elements for focusing a microorganism engineering project might be those that exist on a metabolic pathway that results in production of the compound, e.g., the proteins in the pathway, particularly proteins that catalyze production of the compound or an intermediate. Of course, less obvious elements might also have a significant impact.

In general, it is desirable to frame the microorganism engineering effort to focus resources on impactful edits; there is no use making edits that are unlikely to improve a strain such as making it be more productive or increasing its yield.

In certain embodiments described herein, a microorganism engineering project employs a vector representation of a biological network. The vector representation allows researchers to input elements known to have a direct impact on a goal of the project (e.g., an element in the metabolic pathway for generating a compound of interest). Within the vector representation, other biological elements are positioned with respect to an input element based on biological relatedness. In this manner, based on positions within the vector representation, candidate elements may be identified for editing. These related elements, as well as the input element, may be targeted for editing or other action. For example, if the elements are associated with a gene or operon, the gene sequence may be mutated and/or a different promoter may be used for regulating the gene.

Various aspects of this disclosure pertain to biological networks and their presentation in vector representations that embed or otherwise account for biological relatedness of nodes (representing biological elements) in the biological network. In certain embodiments, biological networks are represented as graphs of nodes such as proteins, reactions, compounds, genes, and other biologically-relevant elements. Such graphs are converted to vector representations in which the nodes are presented at positions in the vector space based on their biological relatedness. Various mathematical techniques may be employed to convert graphs of a biological network into such vector representations. Some such techniques may involve finding a real-valued representation of the nodes that faithfully matches, to a point, the network representation. However, while the vector representation encodes information about the biological network, and it does so in a way that may better reflect biological relatedness between nodes than in a graph of the biological network.

After a vector representation has been generated, it may be “queried” using a query node (input element) of interest, which may be a node or element in the biological network that has some biologically interesting or biologically relevant property for purposes of the engineering project such as a gene of a metabolic pathway that is central to a function to be improved. The query node may be present in the vector representation at a particular location. Based on this location, the system may identify another node or a prioritized set of other nodes and, optionally, associated edits of those nodes.

Biologically related nodes for editing may be identified in the vector representation using various techniques. For example, using separation distance, angle, or other geometric indicator of biological relatedness between a query node and other biological nodes in the vector representation, particular nodes may be selected. In some cases, overall rankings and/or sampling probabilities for node edits may be created. For example, the vector representation may be used to develop sampling probabilities of genes to edit.

There may be multiple phases of a microorganism engineering project, and over the course the project, data from assays may gradually become available. Such data may be useful for supervised training to facilitate or guide further exploration of nodes space for editing or other modification. However, at least initially when very little data is available, methods and systems such as those described herein are particularly useful for recommending elements for editing.

In some cases, a first workflow generates the vector representation for a given biological network, and a second workflow uses the vector representation to identify nodes to edit when considering a microorganism engineering project. In some implementations, the second workflow simply identifies nodes for editing. Then it is up to another resource such as an engineering team using its own knowledge and intuition to select appropriate edits. Alternatively, a different tool or workflow can be used to identify those edits. And, in some cases, the second workflow itself identifies one or more possible edits for a node it considers. Examples of node edits include promoter choices, gene sequence mutations, and gene knockouts. The vector representation in the first workflow may be used repeatedly for multiple different queries (second workflows), each related to the biological network.

In one example, a vector representation is used to identify and rank each or multiple promoter/gene combinations. Stated another way, using information contained in the vector representation, the second workflow identifies promoters to swap in place of existing promoters for particular genes. In some cases, this is done in two operations:
1. Based on distances from the query node for each of multiple genes, a computational system generates a prioritized list or sampling probabilities of the genes.
2. For some or all of the genes identified in 1, the computational system uses a measure of angle (e.g., cosine similarity) or some other measure to generate a prioritized list or sampling probabilities of multiple promoters for the gene.

In some cases, a probability vector is sampled to generate the desired number of strain edits.

Challenges

Graph distance is a common way of finding nodes that are likely to be related. For example, two nodes that are connected have a graph distance of one. Given that two nodes are connected, they are related in some way, and the neighbors of neighbors are likely related to a particular node. However, in the case of so-called “small-world” networks, some nodes behave as hubs and are highly connected and provide what may be viewed as short circuits between biologically unrelated nodes. This means that graph distance may be misleading or even largely meaningless because the distribution of edge distances is small, and many nodes share the same pairwise edge distances. This phenomenon shows up in biological networks quite often. For example, common nodes in a metabolic network, such as water or certain cofactors, produce small graph distances between fairly unrelated nodes in a metabolic network, thus rendering graph distance an ineffective method for finding similar nodes, i.e., nodes that have a biological similarity. Certain heuristics may be used to provide a more meaningful measure of biological relatedness over a graph, but such heuristics introduce biological considerations—e.g., considering which nodes to exclude—and concomitant software engineering challenges.

Certain embodiments disclosed herein overcome this challenge by using node embeddings, which provide a representation having more meaningful relationship between node separation and biological relatedness. In certain embodiments, a graph of a biological network is converted to a vector representation having such embeddings. Distance metrics in such representation can give more precise measures of biological relatedness than graph distance. This enables, for example, more useful ranking of nodes without requiring heuristics for removing cofactors or other common elements (potential hubs) in a biological network.

Another challenge for ranking potential nodes to edit and/or edits themselves is that some supervised methods require a measured phenotype output such as the generated biomass. In genome-wide association studies, for example, it is common to perform regression on the generated biomass based and genome edits. While this approach may be useful in later stages of an engineering program, it is not useful or even available at the beginning stages, as there may be no or minimal assay measurements.

Certain disclosed embodiments overcome this challenge by using known biological networks, such as metabolic networks or a gene ontologies, as inputs and determine node similarity to identify potential edits. This may be a largely unsupervised method.

Another challenge when identifying edits is that in a strict ordering based on biological relatedness may generate too many similar edits clustered together. For example, if two genes of related function are similar and thus have similar ranks, then sampling in the strict rank order will yield less information about the set of possible hits than a more exploratory sampling plan.

Certain embodiments overcome this challenge by converting the distance or similarity between nodes into sampling probabilities. This helps ensure greater exploration over a set of possible edits. Moreover, a tuning hyperparameter may be used to shift the probabilities from being relatively uniform to being sparser.

### Terminology

Terms used in the claims and specification are defined as set forth below unless otherwise specified.

The term “biological network” represents a set of biological and, in some cases, physical processes that influence or determine the physiological and biochemical properties of a cell. In some cases, a biological network includes a partial or complete set of metabolic processes of a cell. In various embodiments, a biological network includes the chemical reactions of metabolism, the metabolic pathways, and may include regulatory interactions that guide these reactions. A metabolic pathway is a linked series of chemical reactions occurring within a cell. The reactants, products, and intermediates of an enzymatic reaction are known as metabolites, which are modified by a sequence of chemical reactions catalyzed by enzymes.

The term “graph” refers to a logical structure containing a set of objects in which some pairs of the objects are in some sense “related.” The objects correspond to mathematical abstractions called nodes (also called vertices or interacting elements) and each of the related pairs of vertices is called an edge (also called an arc or line). Often, a graph is depicted in diagrammatic form as a set of dots for the nodes, joined by lines or curves for the edges. Each edge may be directed or undirected. An “N-partite graph” is a graph having N sets of nodes such that no edge has two nodes belonging to the same set. Examples of nodes for a biological network include genes, gene ontologies, proteins, compounds, reactions, pathways, transcriptional units, and the like.

The term “biological relatedness” refers to the how closely two elements of a biological system are related. Relatedness may be expressed in terms of the degree to which a change in one element impacts the function of separate element. Examples of changes to an element are the edits discussed herein such as gene promoter swaps, gene mutations, gene knockouts, etc. When such change in a first element strongly affects the functioning of a second element, e.g., the second element's rate of converting a first compound to a second compound greatly increases, the first and second elements may be said to have a high degree of biological relatedness. By contrast, when such change in the first element has little effect on the second element, the first and second elements have a relatively low degree of biological relatedness.

The term “vector representation” refers to a mathematical representation of a physical system such as a biological network of a microorganism that presents elements of the physical system at locations that can be described in terms of vectors. In certain embodiments, a vector representation serves a “model” of a microorganism (or group of microorganisms) in that it presents elements of the microorganism in way that indicates their biological relatedness. In certain embodiments, locations of nodes or other points in the vector representation are real-valued.

A vector representation may have an origin and various points, each having a distance from the origin. A vector representation may have axes and various lines or paths that have angles with respect to such axes. The distances and/or angles may be determined or characterized by vectors, and, as indicated, the vectors may represent physical elements, e.g., genes, proteins, reactions, ontology terms, and/or pathways in microorganisms.

In certain embodiments, a vector representation indicates biological relatedness of elements in a microorganism, which elements are represented as nodes or vectors in the representation. The biological relatedness of two elements in vector representation may be indicated by separation distance, angle, or other geometric indication of separation between the two elements. As such, a vector representation may be used identify candidate elements for editing. In some cases, the candidates are determined to be biologically related to some element known to be important to a particular function of the microorganism.

A vector representation may be provided in a Euclidean space or a non-Euclidean space. One example of a non-Euclidean space is a hyperbolic space. One example of a hyperbolic space is one that uses a Poincare representation. Mathematically, the vector space may be represented as v ∈ [symbol] ⁿ where n in the dimensionality of the space. The vector space is fit to the network such that close vectors in [symbol] ⁿ are biologically related.

Generating a Vector Representation from a Graph

Aspects of this disclosure pertain to vector representations that include nodes of a biological network and methods of generating such vector representations from biological networks. In certain embodiments, biological networks are provided as graphs. A vector representation provides an improved representation compared to a simple graph of a biological network for establishing the biological relatedness of nodes, at least as such nodes are relevant to a microorganism engineering project.

Before proceeding, certain concepts of a graph representation of a microorganism will be discussed. In principle, the graphs and vector spaces described herein can be applied to any organism including single celled organisms and higher organisms. In certain embodiments, the organism is a microbe that is naturally incapable of or has limited capability for production of the molecule of interest. In some embodiments, the microbe is one that is readily cultured, such as, for example, a microbe known to be useful as a host cell in fermentative production of molecules of interest. Bacteria, including gram positive or gram negative bacteria can be engineered. For convenience, further discussion will focus on microorganisms, and particularly strains of a single species. Often such species will be used for industrial production of particular chemical compounds.

Generally, a graph includes a biochemical and/or genetic representation of an organism. As explained, the graphs can have nodes (sometimes referred to in the art as vertices or points) and edges connecting the nodes. The nodes represent biochemical or genetic aspects that are relevant to a property of interest for an organism (e.g., the organism's ability to produce a chemical compound in an industrial setting). Any given edge in the graph connects any two nodes. Edges may be directed or undirected and are relevant to the biochemical or genetic aspects of the nodes. They may represent different types of relationships. For example, an edge may connect an enzymatic reaction (first node) to a chemical product of the reaction (second node). If the reaction is generally irreversible, the edge is directed. In another example, directional edges are drawn between a gene and a protein that is translated from the gene. In certain embodiments, graphs are directed, n-partite graphs.

In certain embodiments, the graph includes two or more types of nodes (e.g., genes and reactions or reactions and chemical reactants). In certain embodiments, the graph includes two or more types of edge (e.g., reactions from genes and chemical products from reactions). Further examples will be provided elsewhere herein. Complex graphs, such as these, that have different types of nodes and/or edges are sometimes referred to in the art as knowledge graphs.

Examples of biological systems or networks represented by graphs include metabolic networks and gene interaction networks of an organism. In certain embodiments, nodes and edges of the graph are fixed from strain-to-strain for a type of microorganism, but features of the nodes may vary. This provides a consistent representation for capturing strain edits represented.

In the case of a metabolic network graph, the nodes may include, for example, the graph may include nodes representing one or more genes, one or more chemical species (reactants and/or products), and one or more metabolic reactions. That is, there are different node types for genes, reactions facilitated (e.g., catalyzed) by gene-encoded proteins, and chemical compounds that are reactants or products of the reactions. In this example, the graph edges include reaction connections between reaction nodes and product (metabolite) nodes, reaction connections between reactant (metabolite) nodes and reaction nodes, and gene connections between gene nodes and reaction nodes. Additional biological information may be incorporated in form of additional relationship types (e.g., activator/repressor relationships between gene nodes). Additional nodes may include gene ontology characteristics, transcriptional factors, pathways, RNA, operons, homologs/paralogs/orthologs, etc.

While typically not required, some or all of the nodes may be provided with associated features, some of which may represent edits to a base strain and some of which may represent common properties of a node (regardless of editing). Examples of features include gene structure (e.g., amino acid sequence), gene ontologies, gene promoters, gene product molecular weight, reaction types (e.g., standard reaction classifications such as the Enzyme Commission number and coenzymes required), and chemical structural features (e.g., number of atoms of different types (each of carbon, oxygen, nitrogen, sulfur, etc.) in the compound, bond types, pharmacophore features, and conformations). In one example, the features of a gene include multiple gene ontology classes (e.g., molecular function, cellular component(s) where the gene products are active, biological processes and/or pathways where the gene products are active, etc.), multiple promoter options, and multiple modification types. Note that the process of generating a vector representation of a graph may effectively generate or identify “features” of nodes of the graph and that such features may be incorporated in a variant graph useful for other purposes such as graph neural networks. See e.g., U.S. patent application Ser. No. 16/053,679, filed Aug. 2, 2018, which is incorporated herein by reference in its entirety.

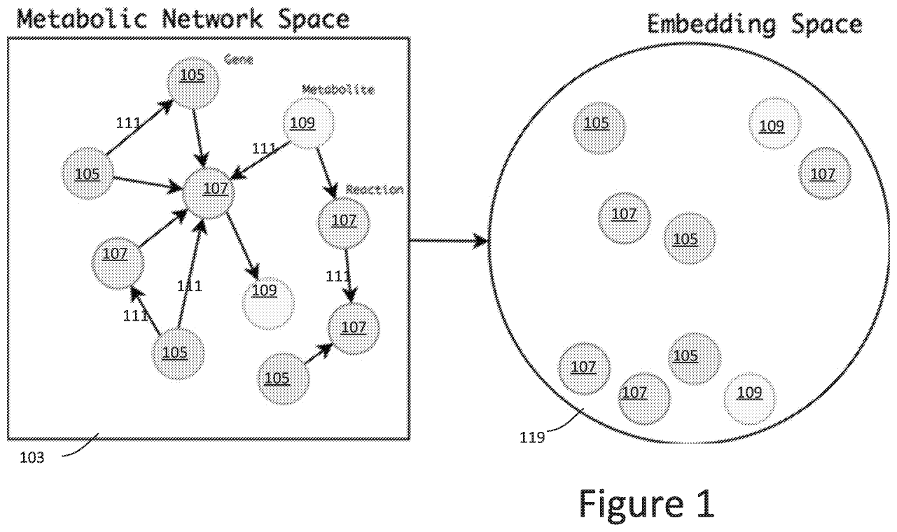

As mentioned, a vector representation of a biological system such as a metabolic network contains node information present in a graph of the system but the nodes are separated by distances that better reflect their biological relatedness. A simple schematic example of the graph and vector space relationship is presented in FIG. 1, where a metabolic network graph 103 contains gene nodes 105, reaction nodes 107, and metabolite (compound) nodes 109. These nodes are connected by edges 111 (only some are labeled in the figure). A vector space 119, which may be derived from the graph 103, contains distinct locations for the gene nodes 105, the reaction nodes 107, and the metabolite nodes 109. However, these node locations have been determined to account for the biological relatedness of the nodes.

In certain embodiments, a graph embedding method receives an input edge list or representation of a graph and generates a real-valued vector representation, where position in the vector space encodes biological information. The resulting vector space can be used to generate the associated vectors for each node.

Various methods may be employed to embed graph nodes in a vector space. Two examples will be presented here.

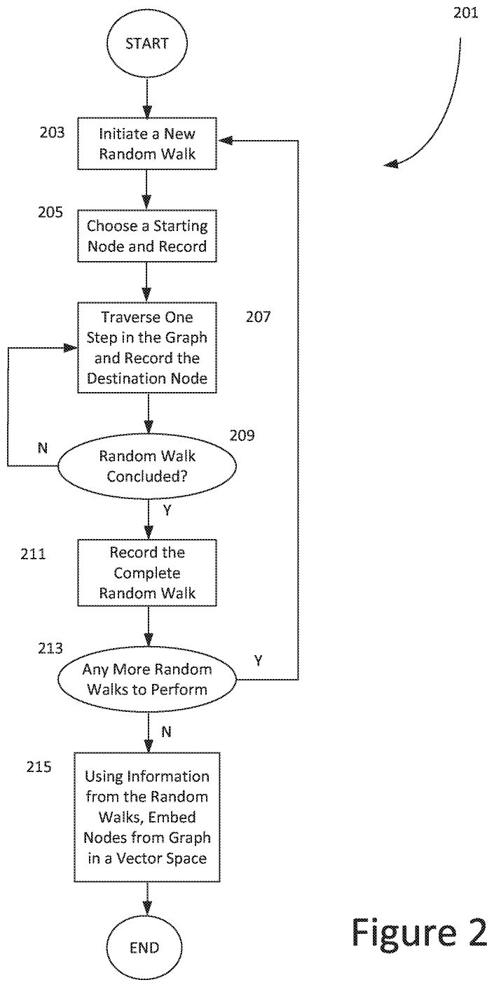

A first method involves a search for nodes that are traversed frequently or in multiple biological contexts (e.g., across different pathways) and effectively deemphasizes them in the vector representation. These nodes may be, for example, hub nodes in small world networks. FIG. 2 illustrates an example process flow 201 for conducting this approach to generating a vector space.

The search involves performing multiple random walks though nodes in a graph of a biological network. Each such walk starts at a first node, takes a first step across an edge to a second node adjacent to the first node, a second step across an edge to a third node adjacent to the second node, and so on. The walks may be fully random or have a limited bias.

As depicted process flow 201 begins with an operation 203 that involves initiating a new random walk. Before beginning the random walk, the embedding method first chooses a starting node in the graph. See operation 205. In certain embodiments, the method chooses the starting node randomly. With the starting node selected, the random walk can commence by taking a first step across an edge connected to the starting node of the graph. See operation 207. The step is randomly chosen from among all the possible steps available to the starting node. For example, if there are three edges connected to the node, each edge may have a 33% chance of being selected for the step. Regardless of how the step is chosen, the destination node is recorded. The stepping and recording continues until the random walk is completed; e.g., until a defined number of steps is taken. As illustrated in FIG. 2, the embedding process may be configured with a check or counter that determines after each step whether the current random walk is concluded. See operation 209. Ultimately, the process determines that the random walk has concluded and records the complete random walk. See operation 211. At this point, another random walk may begin, but first the process determines whether all the required random walks have been completed. See operation 213. Assuming that one or more additional random walks are needed, the process loops back to operation 203, where a new random walk is commenced. While these walks may be purely random, this need not be the case. For example, some level of bias may be applied to the random walks.

Further, the process may include a heuristic to sample each node at least once to ensure low-connected nodes are ultimately embedded.

When all random walks are concluded, the process uses the node lists for each random walk to identify positions for nodes in the vector space. See operation 215.

The resulting node positions account for the distinct pathways in which some nodes occur and the ubiquitous nature of other nodes. The resulting vector space may be a Euclidean space.

In various embodiments, a random walk involves walking a set distance, e.g., 20 steps, many different times. In one example, the random walk involves 20 steps and it is repeated 10,000 times. One way to represent the results of this exercise is to provide a matrix of 10,000 rows and 20 columns. At each location in this matrix, a particular node occupied on the step is provided. The embedding method uses this information to construct the vector space. As an example, the matrix may be evaluated to determine how frequently and/or in what context particular nodes are reached during the random walks. Those nodes that are reached frequently and/or in multiple contexts are deemphasized, as they are moderately close to many clusters for a certain context. Examples of random walk embedding approaches are described by A. Grover and J. Leskovec in “node2vec: Scalable Feature Learning for Networks,” KDD 2016, Aug. 13-17, 2016, San Francisco, Calif. (ISBN 978-1-4503-4232-2/16/08), by B. Perozzi et al. in “DeepWalk: Online Learning of Social Representations,” arXiv:1403.6652v2 [cs.SI] 27 Jun. 2014, and by Nickel et al. in “Poincare Embeddings for Hierarchical Representations,” arXiv: 1705.08039v2 [cs.AI] 26 May 2017.

The embedding method accounts for close node relationships found in the graph. For example, two interacting genes on the same metabolic pathway will frequently occur in the same random walk, and, in those random walks in which they both occur, they will be relatively close to one another. This information may be used to position the nodes close to one another. The embedding predicts whether a given node is in a given pathway based on the results of the random walks.

Nodes such as water that occur in multiple contexts may be deemphasized in the vector space. As an example, one part of the graph may represent an amino acid synthesis pathway, and as a result some fraction of walks are centered around nodes of the amino acid synthesis pathway; another part of the graph is a transporter pathway, resulting in some fraction of the walks centered around transporter activity. However, water or another hub node might occur in both of these pathways, and therefore frequently appear in random walks for both pathways. Therefore, because water appears in multiple contexts, it is relatively meaningless for predicting biological similarity, and can be deemphasized in the vector space. One way to deemphasize such nodes is to place them at locations intermediate to locations different types of nodes (e.g., locations of amino acid synthesis nodes versus locations of transporter nodes), without affecting the distance between the locations of the different types of nodes. Other node types are relatively meaningless compared to highly specific and high impact biological elements, but yet relatively important compared to water. Examples include generic precursors for, e.g., various amino acids.

A second approach to generating the vector representation involves accounting for a hierarchical nature of the nodes in the graph. The resulting vector space may be a non-Euclidean space such as a hyperbolic space (e.g., a Poincare space). Such non-Euclidean spaces are often amenable to embedding information from graphs. For example, these spaces can more naturally encode hierarchies than Euclidean spaces; therefore distances between two points in a hyperbolic space such as a Poincare space can accurately represents distances in a graph of a biological system.

In some cases, a hierarchy may be viewed as a tree with root, trunk, branch, and leaf components. The leaf components may be nodes, particularly nodes that are highly specific (e.g., not shared across multiple contexts) in a biological system. These nodes are generally positioned near an outer edge or perimeter of the space. As an example, a relationship between a leaf node on the left side of a vector space (e.g., the side is associated with a first pathway or context) and a leaf node on the right side of the vector space (e.g., a second pathway or context not clearly related to the first pathway or context) cannot be found directly. E.g., the relationship in the vector space cannot be obtained by directly proceeding from leaf-to-leaf, point-to-point. Instead, the relationship is discerned by traversing from a left leaf node, down trunk/root of the hierarchical structure in the vector space, and then out to the leaf node on the right. Hyperbolic spaces naturally present paths through such hierarchies to get between nodes.

As a further illustration, water as a node typically has many edges and is connected to a many different nodes in different contexts (e.g., different metabolic pathways). Frequently to move between nodes, in different contexts, a graph path traverses through water. In a hyperbolic space, nodes, such as water or certain cofactors, with high centrality or many edges are positioned close to middle origin of the hyperbolic space (which may be viewed as part of a trunk of the hierarchy), while nodes with fewer edges are positioned closer to the edges of the space. In certain embodiments, nodes close to the origin of a hyperbolic space are separated from their adjacent nodes (as represented in the graph) by smaller distances than are nodes in the periphery of the hyperbolic space. As a consequence, edges or hops between centrally located nodes in a hyperbolic space are shorter than edges or hops between peripherally located nodes in the hyperbolic space.

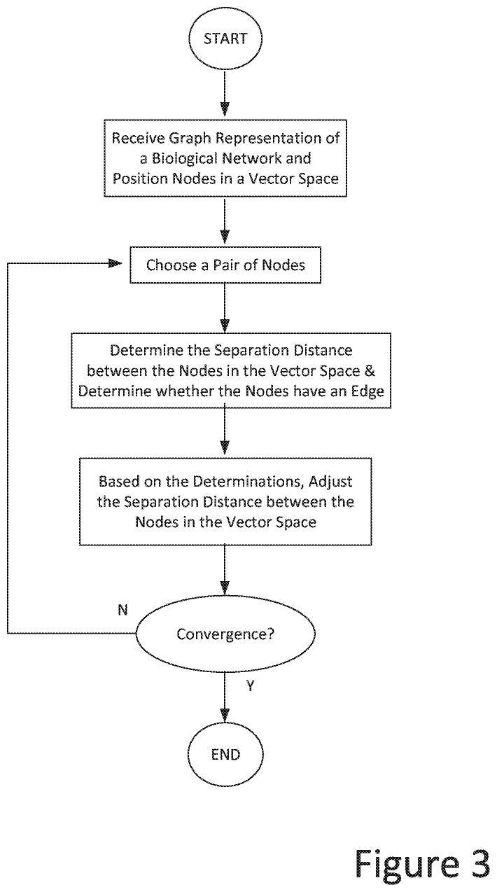

In some cases, a process for converting a graph representation of a biological system to a hyperbolic vector space representation provides an initial distribution of node placements in the hyperbolic space. It then determines whether there are edges between pairs of nodes in the hyperbolic space. For example, using two points in the initial distribution, the embedding method may try to predict whether those two points are connected by an edge in the original graph. If the nodes are close together in both the hyperbolic space and are connected by an edge in the graph, the embedding method interprets this as a positive result and little effort is made to relocate the points with respect to one another. However, if the two nodes are unconnected by an edge in the graph, there is a penalty for keeping them close together in the vector space. Hence, they may be moved further apart in the vector space. And, if two nodes are by an edge in the graph but far apart in the vector space, there is a penalty for keeping them far apart in the vector space. This embedding process is conducted iteratively using pairs of nodes in the vector space, moving nodes closer together or further apart depending on whether one or more hops exist between them in the graph. Convergence checks are employed to determine when node positioning in the vector space is complete. See as an example, FIG. 3.

When implementing the embedding process, the nature of the hyperbolic space naturally accounts for the underlying hierarchy in the graph structure. In the process, water and other relatively less useful nodes are pushed toward the middle of the vector space.

An example Poincare embedding approach is described by M. Nickel and D. Kiela in “Poincare Embeddings for Learning Hierarchical Representations,” arXiv:1705.08039v2 [cs.AI] 26 May 2017.

Multiple Organisms

In certain embodiments, multiple organisms may be modeled in a single graph, and hence in a single vector space. The nodes in the network represent biologically relevant information such as compounds or genes, which are found in multiple related organisms, e.g., multiple strains of a microorganism. In other words, these nodes may be conserved across organisms, for example certain proteins have the same enzymatic function even if they are in two different organisms. In this framework, the networks from two different networks can be combined. For example if ADP is synthesized in both organisms, then a single node with edges relevant to both organisms can be put into the model.

Distance or Biological Relatedness in a Vector Representation

In certain embodiments, the vector representation has an origin, angles, and distances with respect to the origin. As indicated, the vector space may be a Euclidean space or a non-Euclidean space.

In a graph representing a biological system, distance between two nodes is easily represented as the number of edges or hops between the two nodes. In vector representations, however, distance is not limited to a number of edges or hops between nodes, and, in fact, a uniform distance per hop can be detrimental. In the vector space, various measures of distance may be considered. In general, any such measure provides some indication of a separation between nodes, and the separation is not dictated by number of hops in a graph. The separation may be determined along a linear path, a curved path, an angle, etc. As examples, distances along a line may be determined in a Euclidean space, distances along curves may be determined in a non-Euclidean space, and angles, trigonometric values, etc. may be determined in Euclidean or non-Euclidean types of space.

In some approaches, distance is determined by first, for each of the nodes, creating a separate vector from the origin of the vector space to the location of the node. In a particular example, angular separation is determined using a cosine similarity. In certain embodiments, a vector space created from a graph does not have dimensions or units that are directly translatable to physical parameters such biological parameters.

One example of a non-Euclidean space is a hyperbolic space. One example of a hyperbolic space is one that uses a Poincare representation.

In a hyperbolic space, two nodes on the same general radial trajectory, are commonly on the same pathway in a graph (e.g., on the same metabolic pathway). And, on such pathway, nodes located relatively closer to the origin may be upstream from nodes relatively closer to the perimeter. For example, a terminal node in a pathway may be close to the perimeter of the vector space, while a node for producing an early precursor may be close to the origin.

Using the Vector Representation to Identify Nodes for Editing (Microorganism Engineering)

In certain embodiments, a vector space is used to identify nodes for editing in strain engineering projects, particularly projects where little if any assay data is available for indicating how particular genes, proteins, etc. impact a goal of the project. For example, little if any information may be available about whether certain gene-promoter combinations are likely to improve strain performance.

A process of choosing nodes to edit in a microorganism engineering project may involve initially selecting a particular input entry, which is a node in the biological network that has some biologically interesting or biologically relevant property for purposes of the engineering project. Examples of such inputs include genes, pathways, and proteins, but any type of node in the vector representation may be used. In various embodiments, an input node or query node is chosen because it is directly related to the goal of a strain engineering project, e.g., it is a terminal protein or pathway including the protein that catalyzes a reaction that is involved in the generation of a goal metabolite whose enhanced production is a goal of a strain engineering project.

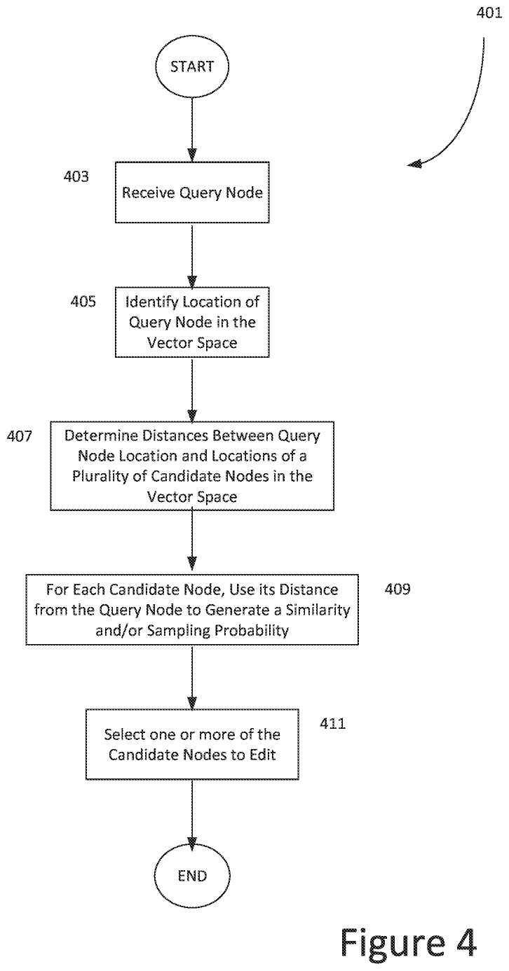

When that node or input entry is selected, it is located or positioned in the vector representation and the sequence of operations takes place to allow the user or team responsible for engineering to pick a prioritized set of nodes for editing. FIG. 4 provides an example of the process. As illustrated, a process 401 begins by receiving the identification an input or query node (e.g., the last protein in a pathway for producing a compound of interest). See operation 403. The query node is then located in a vector space, such as one of those described above, that positions nodes based on their biological relatedness. See operation 405. Typically, the node was positioned in the vector space when the vector space was created, so operation 405 simply involves finding the pre-defined location of the query node in the vector space. Next, the process determines a distance in the vector space between the query node and a first candidate node, i.e., one of the other nodes from the biological system that have been embedded in the vector space. The distance and the identification of the candidate node may be saved.

The process similarly determines distances in the vector space between the query node and each of a plurality of other candidate nodes in the vector space. See operation 407. The candidate nodes may be proteins, genes, pathways, gene ontology parameters, etc. that were embedded in the vector space. In some approaches, each node of a particular type in the vector space serves as a candidate node. The distances may be linear distances in, e.g., a Euclidean space, curves in a non-Euclidean space, angles, trigonometric values, etc. At the conclusion of operation 407, the process has generated a list of distances associated with each of the candidate nodes. These distances may be used as is to determine which candidate nodes are edited first. For example, the node closest to the query node may be edited first. In alternative embodiments, the distances are not used as such but are first converted to similarity values and/or sampling probabilities. See operation 409. Using such values, candidate nodes may be ranked or selected for editing. See operation 411. In various embodiments, candidate nodes are sampled without replacement.

In some examples, the sampling probability for each gene or other node is generated by the following sequence.

1. Choose a query node such as, e.g., a terminal gene in a pathway.

2. Determine distances from the query node to each other possible candidate nodes in the vector space. This may be accomplished by first creating a vector for each node (e.g., a vector from the origin of the space) and then determining distances between the query node vector and each of the other vectors.

3. Convert distances to sampling probabilities
Option 1—convert distances directly to probabilities
Option 2—first convert distances to similarities and then convert the similarities to probabilities. One reason to first generate a similarity is because nodes close together in vector space, and that are therefore very similar, would, assuming a direct conversion to probability, be sampled at a disproportionately lower rate than would nodes that are far apart. In other words, shorter distances correspond to relatively higher similarity. A function for converting distance to similarity may produce similarity values that are between 0 and 1. Using, for example, 1/(1+distance) as a measure of similarity, nodes that are very close to the query node all have a high value of similarity.

In certain embodiments, generation of probabilities is conducted in a way that normalizes all the similarities into probabilities and the probabilities sum to a value of 1. One example of a suitable probability generation function is the softmax function, which takes a non-normalized vector, and normalizes it into a probability distribution. Prior to applying softmax, some vector elements could be negative, or greater than one; and the elements might not sum to 1; but after applying softmax, the elements sum to 1.

Hyperparameters can be set to control how much exploration of nodes far away from the query node is to be performed. Some hyperparameter values bias the probability values to limit exploration to candidate nodes that are close to the query node, while different hyperparameter values skew the probability values to expand exploration to candidate nodes that are further away from the query node, i.e., they tend to cause wider exploration. To promote wider exploration, the probability values of the candidate nodes may be made more uniformly separated than they would be otherwise. In the softmax function, a hyperparameter known as the “temperature” parameter may be adjusted to permit wider or more focused exploration of the vector space.

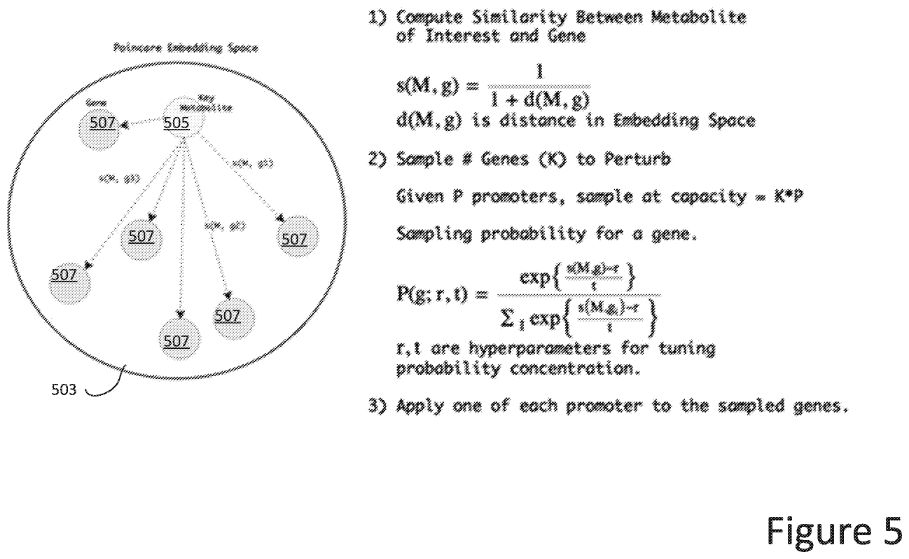

FIG. 5 depicts an example process for creating sampling probabilities from a metabolite in an embedding space. In the example, a Poincare embedding space 503 contains a key metabolite node 505, which serves as a query nodes, a plurality of gene nodes 507, which serve as candidate nodes for editing. Distances (d(M,g)) are generated between key metabolite node 505 and each of the candidate gene nodes 507. These distances are converted to similarities S(M,g) using a reciprocal distance function as shown (S(M,g)=1/(1+d(M,g))). The similarity values are converted to gene sampling probabilities, P, using a function having two tuning hyperparameters, r and t, as illustrated. In the softmax function, the temperature hyperparameter is used.

Genes are then sampled for editing according to their sampling probability values. When a gene is selected through such sampling, it is edited in a prescribed manner. For example, it may be expressed using each of a plurality of promoters, e.g., promoters in a promoter ladder.

Use Vector Space to Identify Edits (Microorganism Engineering)

As indicated, a vector space with embedded nodes from a biological network may be used to simply identify nodes for editing. With this information, a project team may choose particular nodes to perturb based on sampling probabilities generated from the vector space, and then determine how to edit the chosen nodes. However, in some embodiments, the vector space may be used to identify particular edits. Examples of such edits include promoters swaps for a gene, gene knockouts or terminations (simply removing or disabling a gene), sequence edits to a gene such as point mutations (e.g., at SNPs), and other changes. In some cases, the vector space suggests a measure of genetic diversity that may be used in future design of experiments for, e.g., finding new growth conditions for strains.

Locations of nodes in vector space may be used to identify edits (different regions of vector space may be associated with different types of edits). In various embodiments, angular separation is used to identify different types of edits. For example, the direction and strength of edit may be determined from angular similarity (or a resulting sampling probability vector). In such embodiments, angular separation may be used in lieu of point-to-point distances. Such embodiments may use either a Euclidean or non-Euclidean vector space (e.g., a Poincare vector space).

Using an angular separation approach, angular separations between a vector to the query node and candidate nodes are calculated. In some promoter swap embodiments, candidate node vectors that have small angular separations from the query node vector may be more likely to be upregulated. Such nodes may be presumed to be on a pathway having a strong impact on the goal of the strain engineering project. By contrast, nodes that have a large angular separation are more likely to be downregulated; such nodes are less likely to be on the pathway.

In general, assuming that the vector space can be viewed as a circle around the origin, similar nodes that are close together angularly suggest that strong promoters should be used. Candidate nodes separated from the query node by about 90 degrees may have a more neutral regulation, e.g., the strain engineering project would not strongly upregulate or downregulate them.

In general, candidate nodes having vectors directed substantially opposite the vector of the query node will suggest promoter swaps using weaker promoters. In some embodiments, the edit to such nodes is not even a promoter swap to a weak promoter.

Instead, the edit uses a terminator or simply knocks out the gene. The decision on whether to use a weak promoter or a simply knock out the gene may be based on where in an operon the gene is question (the candidate node) is located. For example, if it is at or near the beginning of the operon, it may be more likely to be simply downregulated than terminated or knocked out.

In some embodiments, the angle is not used as such, but is first converted to a cosine or other trigonometric value. If cosine is used, the values of angular similarity span from 1 (most similar) to −1 (least similar). Angle or trigonometric values can be converted to “similarities” and sampling probabilities as described above with respect to distance.

In various implementations, two nodes that have a similar angle are expected to be on the same pathway or otherwise work together. So, for example, two nodes that are on the same radius but at significantly different distances from the origin may be similar in that, e.g., they participate in the same synthetic pathway. From the hierarchy perspective, two nodes on the same general radial path, may be on the same pathway, with the one located closer to the origin being upstream in the pathway compared to one located closer to the perimeter (which node may be, e.g., a terminal node on the pathway).

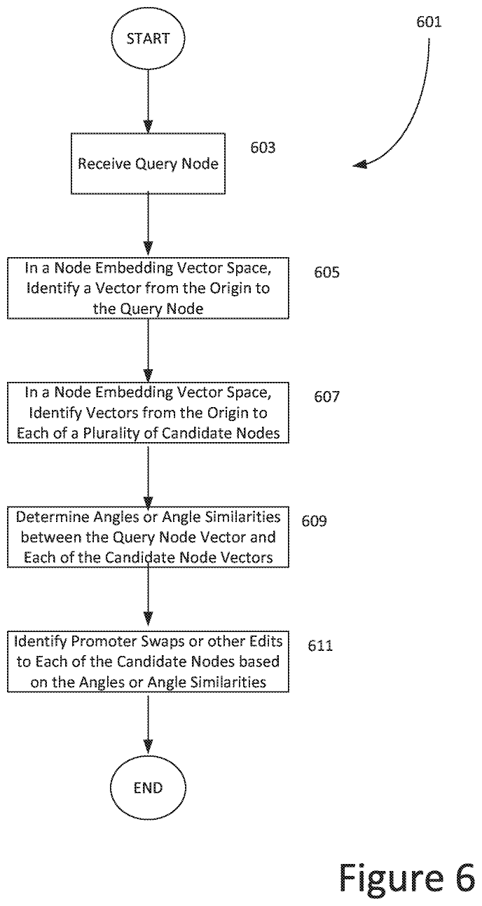

In some embodiments, the promoter swap example is implemented as follows (see for example operations 603, 605, 607, 609, and 611 of process 601 depicted in FIG. 6):
1. Provide promoter ladder of varying strengths.
2. Generate sampling probabilities for each promoter by:
   a) Computing the cosine similarity between the input term and each node in graph
   b) Converting the cosine based similarity to sampling probabilities for the promoters. In some cases, this is accomplished by running the computed similarities through, e.g., the softmax function to generate sampling probabilities, optionally using the temperature parameter or other hyperparameter to distribute sampling probabilities more uniformly.
   c) Partitioning the support of the cosine (angle) space over the possible set of promoters, and generating sampling probabilities dependent on which partition the actual cosine similarity belongs in.
3. Using these sampling probabilities to prioritize a set of possible edits. Based on capacity, the gene/promoter combinations are sampling weighted by the probabilities learned based on the similarities. Sampling may be conducted without replacement.

In certain embodiments, the predicted edits for nodes/elements of a microorganism from an embedded vector space are used to train a machine learning classification model. The resulting model predicts whether a particular edit can be successful. In some implementations the classification model receives a node vector as an input and generates a pass/fail outcome. In certain embodiments, a vector representation of a particular gene from the model is used and a categorical feature represents the promoter. These may be used as input features to the classification model and a 0/1 fail/build score may be given depending on the QC output. In some cases, embeddings may be learned from these models.

System Examples

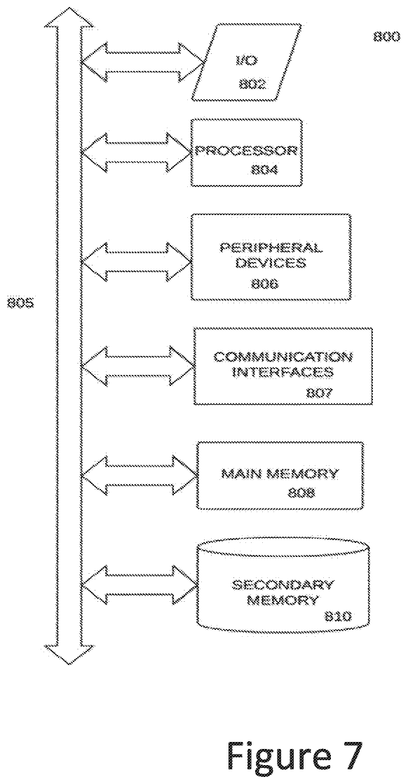

A computer system is typically used to execute program code stored in a non-transitory computer readable medium (e.g., memory) in accordance with embodiments of this disclosure. As depicted in FIG. 7, a computer system 800 may include an input/output subsystem 802, which may be used to implement an interface for interacting with human users and/or other computer systems depending upon the application. For example, the editor of embodiments of the invention may be implemented in program code on system 800 with I/O subsystem 802 used to receive input program statements from a human user (e.g., via a GUI or keyboard) and to display them back to the user. The I/O subsystem 802 may include, e.g., a keyboard, mouse, graphical user interface, touchscreen, or other interfaces for input, and, e.g., an LED or other flat screen display, or other interfaces for output. Other elements of embodiments may be implemented with a computer system like that of computer system 800, but without one or more elements such as I/O subsystem 802.

Program code may be stored in non-transitory media such as persistent storage 810 or memory 808 or both. One or more processors 804 reads program code from one or more non-transitory media and executes the code to enable the computer system to accomplish the methods performed according to the embodiments herein, such as those involved with a graph and vector space embedded with nodes from a biological system as described herein. Those skilled in the art will understand that the processor may receive source code, such as statements for executing embedding and/or querying operations, and interpret or compile the source code into machine code that is understandable at the hardware gate level of the processor. A bus couples the I/O subsystem 802, the processor 804, peripheral devices 806, memory 808, and persistent storage 810.

Those skilled in the art will understand that some or all of the elements of embodiments of this disclosure, such as generating and processing graphs of metabolic networks, producing embedded vector spaces, and querying such vector spaces to identify biological nodes for editing and/or to identify specific edits may be implemented wholly or partially on one or more computer systems including one or more processors and one or more memory systems like those of computer system 800. Some elements and functionality may be implemented locally and others may be implemented in a distributed fashion over a network through different servers, e.g., in client-server fashion, for example.

High throughput libraries methods and systems for automating the design and construction of genetic elements to produce modified cells are known to those of skill in the art. Such techniques can be employed to produce modified microorganisms having modifications selected or identified by the techniques described herein. In some implementations, these techniques employ microbial genomic engineering for controlling production of nucleotide sequences by a gene manufacturing system. In certain embodiments, a laboratory information management system is used for building, testing, and analyzing DNA sequences and/or engineered microbe genomes. Such system may integrate a microbe design phase implemented using models such as the vector spaces embedded with nodes from a biological network described herein.

Some pertinent high throughput methods and apparatus are described in US Patent Application Publication US 2017/0159045, published Jun. 8, 2017, now U.S. Pat. No. 9,988,624, and International Patent Application Pub. No. WO/2017/189784, published Nov. 2, 2017, incorporated herein by reference in their entireties.

In some embodiments, a microorganism is engineered or otherwise modified based on information obtained from the embedded vector space. The microorganism may be used to produce a desired molecule. A microbe that produces the molecule of interest (either naturally or via genetic engineering) can be engineered to enhance production of the molecule. In some embodiments, this is achieved by increasing the activity of one or more of the enzymes in the pathway that leads to the molecule of interest. In certain embodiments, the activity of one or more upstream pathway enzymes is increased by modulating the expression or activity of the endogenous enzyme(s). Alternatively or additionally, the activity of one or more upstream pathway enzymes can be supplemented by introducing one or more of the corresponding genes into the microbial host cell. For example, the microbe can be engineered to express multiple copies of one or more of the pathway enzymes, and/or one or more pathway enzymes can be expressed from introduced genes linked to particularly strong (constitutive or inducible) promoters. An introduced pathway gene may be heterologous or may simply be an additional copy of an endogenous gene. Where a heterologous gene is used, it may be codon-optimized for expression in the particular host microbe employed. Any or all of these modifications may be recommended using embedded vector spaces to evaluate many possible putative modifications.

The term “engineered” is used herein, with reference to a cell, to indicate that the cell contains at least one genetic alteration introduced by man that distinguishes the engineered cell from the naturally occurring cell.

Any microbe that can be used to express introduced genes can be engineered for fermentative production of molecules. In certain embodiments, the microbe is one that is naturally incapable fermentative production of the molecule of interest. In some embodiments, the microbe is one that is readily cultured, such as, for example, a microbe known to be useful as a host cell in fermentative production of molecules of interest. Bacteria cells, including gram positive or gram negative bacteria can be engineered as described above. Examples include C. glutamicum, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulars, B. lautus, B. thuringiensis, S. albus, S. lividans, S. coelicolor, S. griseus, P. citrea, Pseudomonas sp., P. alcaligenes, Lactobacilis spp. (such as L. lactis, L. plantarum), L. grayi, E. coli, E. faecium, E. gallinarum, E. casseliflavus, and/or E. faecalis cells.

The term “fermentation” is used herein to refer to a process whereby a microbial cell converts one or more substrate(s) into a desired product by means of one or more biological conversion steps, without the need for any chemical conversion step.

There are numerous types of anaerobic cells that can be used as microbial host cells in the methods described herein. In some embodiments, the microbial cells are obligate anaerobic cells. Obligate anaerobes typically do not grow well, if at all, in conditions where oxygen is present. It is to be understood that a small amount of oxygen may be present, that is, there is some level of tolerance level that obligate anaerobes have for a low level of oxygen. Obligate anaerobes engineered as described above can be grown under substantially oxygen-free conditions, wherein the amount of oxygen present is not harmful to the growth, maintenance, and/or fermentation of the anaerobes.

Alternatively, the microbial host cells used in the methods described herein can be facultative anaerobic cells. Facultative anaerobes can generate cellular ATP by aerobic respiration (e.g., utilization of the TCA cycle) if oxygen is present. However, facultative anaerobes can also grow in the absence of oxygen. Facultative anaerobes engineered as described above can be grown under substantially oxygen-free conditions, wherein the amount of oxygen present is not harmful to the growth, maintenance, and/or fermentation of the anaerobes, or can be alternatively grown in the presence of greater amounts of oxygen.

In some embodiments, the microbial host cells used in the methods described herein are filamentous fungal cells. (See, e.g., Berka & Barnett, Biotechnology Advances, (1989), 7(2):127-154). Examples include Trichoderma longibrachiatum, T viride, T koningii, T harzianum, Penicillium sp., Humicola insolens, H lanuginose, H grisea, Chrysosporium sp., C. lucknowense, Gliocladium sp., Aspergillus sp. (such as A. oryzae, A. niger, A. sojae, A. japonicus, A. nidulans, or A. awamori), Fusarium sp. (such as F. roseum, F. graminum F. cerealis, F. oxysporuim, or F. venenatum), Neurospora sp. (such as N. crassa or Hypocrea sp.), Mucor sp. (such as M. miehei), Rhizopus sp., and Emericella sp. cells. In particular embodiments, the fungal cell engineered as described above is A. nidulans, A. awamori, A. oryzae, A. aculeatus, A. niger, A. japonicus, T reesei, T viride, F. oxysporum, or F. solani. Illustrative plasmids or plasmid components for use with such hosts include those described in U.S. Patent Pub. No. 2011/0045563.

Yeasts can also be used as the microbial host cell in the methods described herein. Examples include: Saccharomyces sp., Yarrowia sp., Schizosaccharomyces sp., Pichia sp., Candida sp, Kluyveromyces sp., and Hansenula sp. In some embodiments, the Saccharomyces sp. is S. cerevisiae (See, e.g., Romanos et al., Yeast, (1992), 8(6): 423-488). In some embodiments, the Yarrowia sp. is Y. lipolytica. In some embodiments, the Kluyveromyces sp. is K. marxianus. In some embodiments, the Hansenula sp. is H. polymorpha. Illustrative plasmids or plasmid components for use with such hosts include those described in U.S. Pat. No. 7,659,097 and U.S. Patent Pub. No. 2011/0045563.

In some embodiments, the host cell can be an algal cell derived, e.g., from a green algae, red algae, a glaucophyte, a chlorarachniophyte, a euglenid, a chromista, or a dinoflagellate. (See, e.g., Saunders & Warmbrodt, “Gene Expression in Algae and Fungi, Including Yeast,” (1993), National Agricultural Library, Beltsville, Md.). Illustrative plasmids or plasmid components for use in algal cells include those described in U.S. Patent Pub. No. 2011/0045563. In other embodiments, the host cell is a cyanobacterium, such as cyanobacterium classified into any of the following groups based on morphology: Chlorococcales, Pleurocapsales, Oscillatoriales, Nostocales, or Stigonematales (See, e.g., Lindberg et al., Metab. Eng., (2010) 12(1):70-79). Illustrative plasmids or plasmid components for use in cyanobacterial cells include those described in U.S. Patent Pub. Nos. 2010/0297749 and 2009/0282545 and in Intl. Pat. Pub. No. WO 2011/034863.

Microbial cells can be engineered for using conventional techniques and apparatus of molecular biology (including recombinant techniques), microbiology, cell biology, biochemistry, and immunology, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

While many modifications suggested using embedded vector space modelling systems described herein involve genome engineering, such modifications may be supplemented by introducing wholly new genes into microbe. Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods in Enzymology 185, Academic Press, San Diego, Calif (1990).

In some embodiments, vectors may be used to introduce systems that can carry out genome editing, such as TALEN (transcription activator-like effector nuclease), zinc finger, meganuclease, and CRISPR systems. See U.S. Patent Pub. No. 2014/0068797, published 6 Mar. 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F. A., et al., (“In vivo genome editing using Staphylococcus aureus Cas9,” Nature 520(7546):186-91, 2015, Apr. 9, including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Vectors or other polynucleotides can be introduced into microbial cells by any of a variety of standard methods, such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220.

The above-described methods can be used to produce engineered microbial cells that produce, and in certain embodiments, overproduce, a molecule of interest. Engineered microbial cells can have at least 1, 2, 3, 4, 5, 6, 7, 8, 9, 10, or more genetic alterations, any one or more evaluated by using a node embedding vector space as described herein, as compared to a wild-type microbial cell, such as any of the microbial host cells described herein. Engineered microbial cells described in the Examples below have two genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a wild-type microbial cell. In various embodiments, engineered microbial cells can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

The engineered microbial cells can contain introduced genes that have a wild-type nucleotide sequence or that differ from wild-type. For example, the wild-type nucleotide sequence can be codon-optimized for expression in a particular host cell. The amino acid sequences encoded by any of these introduced genes can be wild-type or can differ from wild-type. In various embodiments, the amino acid sequences have at least 0 percent, 75 percent, 80 percent, 85 percent, 90 percent, 95 percent or 100 percent amino acid sequence identity with a wild-type amino acid sequence.

In various embodiments, the engineered microbial cells are capable of producing the molecule of interest at titers of at least 4, 5, 6, 7, 8, 9, 10, 20, 30, 40, 50, 75, 100, 200, 300, 400, 500, 600, 700, 800, or 900 mg/L or at least 1, 1.5, 2, 2.5, 3, 3.5, 4, 4.5, or 5 gm/L. In various embodiments, the titer is in the range of 4 mg/L to 5 gm/L, 10 mg/L to 4 gm/L, 100 mg/L to 3 gm/L, 200 mg/L to 2 gm/L, or any range bounded by any of the values listed above.

Engineered microbial cells of interest can be cultured, e.g., for maintenance, growth, and/or production of the molecule of interest. In some embodiments, the cultures are grown to an optical density at 600 nm of 10-500.

In various embodiments, the cultures produce the molecule of interest at titers of at least 50, 75, 100, 200, 300, 400, 500, 600, 700, 800, or 900 mg/L or at least 1, 1.5, 2, 2.5, 3, 3.5, 4, 4.5, or 5 gm/L. In various embodiments, the titer is in the range of 100 mg/L to 5 gm/L, 200 mg/L to 4 gm/L, 300 mg/L to 3 gm/L, or any range bounded by any of the values listed above.

The term “titer,” as used herein, refers to the mass of a product (e.g., the molecule that microbial cells have been engineered to produce) produced by a culture of microbial cells divided by the culture volume.

Microbial cells can be cultured in a minimal medium, i.e., one containing the minimum nutrients possible for cell growth. Minimal medium typically contains: (1) a carbon source for microbial growth; (2) salts, which may depend on the particular microbial cell and growing conditions; and (3) water.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include lactose and sucrose, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce the molecule of interest, the culture medium can include, and/or be supplemented during culture with, glucose and/or a nitrogen source such as urea, an ammonium salt, ammonia, or any combination thereof.

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass. Cell cultures with engineered microbial cells are often provided in a bioreactor or other production vessel, as known to those of skill in the art.

In general, cells are grown and maintained at an appropriate temperature, gas mixture, and pH (such as about 20° C. to about 37° C., about 6% to about 84% CO2, and a pH between about 5 to about 9). In some embodiments, cells are grown at 35° C. In some embodiments, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that is consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for greater than or about 1, 2, 3, 5, 10, 15, 20, 25, 30, 35, 40, 50, 60, or 70 hours. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some embodiments, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract.

The fermentation methods described herein may include an operation of recovering the molecule produced by an engineered microbial host. As used herein with respect to recovering a molecule of interest from a cell culture, “recovering” refers to separating the molecule from at least one other component of the cell culture medium. In some embodiments, the produced molecule contained in a so-called harvest stream is recovered/harvested from the production vessel. The harvest stream may include, for instance, cell-free or cell-containing aqueous solution coming from the production vessel, which contains the produced molecule. Cells still present in the harvest stream may be separated from the molecule by any operations known in the art, such as for instance filtration, centrifugation, decantation, membrane crossflow ultrafiltration or microfiltration, tangential flow ultrafiltration or microfiltration or dead end filtration. After this cell separation operation, the harvest stream is essentially free of cells.

Further steps of separation and/or purification of the produced molecule from other components contained in the harvest stream, i.e., so-called downstream processing steps may optionally be carried out. These steps may include any means known to a skilled person, such as, for instance, concentration, extraction, crystallization, precipitation, adsorption, ion exchange, chromatography, distillation, electrodialysis, bipolar membrane electrodialysis and/or reverse osmosis. Any of these procedures can be used alone or in combination to purify the produced molecule. Further purification steps can include one or more of, e.g., concentration, crystallization, precipitation, washing and drying, treatment with activated carbon, ion exchange and/or re-crystallization. The design of a suitable purification protocol may depend on the cells, the culture medium, the size of the culture, the production vessel, etc. and is within the level of skill in the art.

### EXAMPLE

The following example employed a network (graph) with the following vertices counts:

{‘protein’: 2564, ‘go_term’: 1440, ‘gene’: 2383, ‘reaction’: 1556, ‘compound’: 1340, ‘pathway’: 584, ‘rna’: 318}

And the following edge counts:

{′protein_reaction′: 2123, ‘go_protein’: 10638, ‘gene_protein’: 2304, ‘reaction_reactant’: 3396, ‘reactant_reaction’: 2883, ‘pathway_pathway’: 533, ‘pathway_reaction’: 852, ‘reaction_rna’: 47, ‘protein_protein_component’: 156, ‘reaction_reaction’: 2858, ‘rna_rna’: 315, ‘reaction_protein’: 251, ‘gene_rna’: 79, ‘rna_reaction’: 55}

For context and as an example, “‘go_protein’: 10638” means that there are 10638 edges where a gene ontology (GO) term node is an edge to a protein node. This graph, or more precisely the edge list, is sufficient to train the underlying embeddings model.

Once that embedding model has been fit, it may be encoded as follows:
>>>g=GraphObject( )
>>>embedding_model=EmbeddingModel(g)>
>>embedding_model.fit(epochs=50)

It can be then used to query similar objects. For example, given the goals of a particular strain engineering program, one such query might be the “L-lysine biosynthesis 1” pathway:
>>>embedding_model.query(“L-lysine biosynthesis 1”)

When this query is run, the top five results from one model are:

| Term (using cgl identifiers) | Similarity |
|---|---|
| Cgl0998 | 0.139027 |
| Cgl2303 | 0.137389 |
| Cgl2881 | 0.137052 |
| Cgl0503 | 0.136409 |
| Cgl2569 | 0.136041 |

In this table, the left values are the identifiers for the nodes in question, the right values are similarity scores which are calculations on the distances such that higher similarities correspond to closer distances in the model (embedding space).

With these identifiers, a program can validate the model by, e.g., comparing with historical performance to see how well the model identifies genes that have been determined to be hits through outside means. For example, here a lasso regression is used with fermentation performance of the strains against a one-hot encoded matrix, where the entry in the matrix is one if the strain contains that change and zero otherwise. This is a fairly standard feature extraction procedure, and as a result these are the top changes with the associated effect size of the change.

| | |
|---|---|
| ncgl0304 | 1.628635 |
| ncgl0471 | 0.443952 |
| ncgl0659 | 3.966996 |
| ncgl0668 | 0.303366 |
| ncgl0767 | 2.244001 |
| ncgl0909 | 0.364090 |
| ncgl0976 | 1.562297 |
| ncgl1133 | 0.728149 |
| ncgl1179 | 0.014353 |
| ncgl1261 | 1.147112 |
| ncgl1262 | 2.108213 |
| ncgl1512 | 0.032658 |
| ncgl1514 | 0.915054 |
| ncgl1948 | 0.410531 |
| ncgl2126 | 1.022140 |
| ncgl2425 | 1.576131 |
| ncgl2480 | 1.741161 |
| ncgl2482 | 1.777971 |

In this list, the left values are the node identifiers, and the right values are the regression coefficients from the lasso regression. The presence of the identifier to the left in the strain corresponds to an increase in performance of the coefficient amount; e.g., a strain with an edit to ncg12482 (the last item) will, on average, have increase in output of 1.78 over a strain that does not have an edit to that node.

One can then consider the number of “hits,” defined as being in the list above, that would be found in the first K recommendations based on this model. Ideally, the model would rank changes of interest higher than random and thus “find” more hits at the same K. To that point, the table below is the number of hits found using two different queries and a random result for comparison at various gradations of K. The information in the second column is the same as that in the first, except that rather than querying with lysC (a gene node in the network) it was queried with “L-lysine biosynthesis” (a pathway node). The third column represents the number of hits one would expect if they were sampled at random.

| | | “L-lysine | |
|---|---|---|---|
| | | biosynthesis” | |
| K/Query | “lysC” (Gene) | (Pathway) | random |
| 50 | 2 | 1 | 0.5 |
| 150 | 3 | 2 | 1.5 |
| 250 | 5 | 4 | 2.5 |
| 500 | 10 | 9 | 5.0 |
| 750 | 12 | 10 | 7.5 |
| 1000 | 13 | 16 | 10.0 |

### CONCLUSION

Unless the word “means” or “step” is used in a claim, the claims herein should not be interpreted as invoking 35 USC § 112(f).

While the present invention has been particularly described with respect to the illustrated embodiments, it will be appreciated that various alterations, modifications and adaptations may be made based on the present disclosure, and are intended to be within the scope of the present invention. While the invention has been described in connection with the disclosed embodiments, it is to be understood that the present invention is not limited to the disclosed embodiments but, on the contrary, is intended to cover various modifications and equivalent arrangements included within the scope of the claims.

1. A method of identifying one or more elements for modification to cause a change in functioning of a microorganism, the method comprising:
(a) receiving a graph representation of a biological network of interacting elements of a microorganism or a plurality of related microorganisms;
(b) converting the graph representation of the biological network to a vector representation having locations of the interacting elements, which locations are derived from positions of said interacting elements in the graph representation;
(c) determining distance relationships between the interacting elements as represented in the vector representation; and
(d) from the distance relationships determined in (c), recommending a subset of the interacting elements for modification in an engineered variant of the microorganism or the plurality of related microorganisms.

2. The method of claim 1, wherein recommending a subset of the interacting elements for modification comprises generating sampling probabilities for selecting at least some of the interacting elements for modification.

3. The method of claim 2, further comprising shifting the sampling probabilities to become more sparse.

4-6. (canceled)

7. The method of claim 1, wherein the graph representation of the biological network is a graph representation of a metabolic network of the microorganism or a plurality of related microorganisms.

8. (canceled)

9. The method of claim 1, wherein the graph representation of the biological network comprises two or more interacting elements selected from the group consisting of genes, compounds, reactions, genetic ontology terms, transcriptional units, and proteins.

10-11. (canceled)

12. The method of claim 1, wherein converting the graph representation of the biological network to a vector representation comprises:
conducting a plurality of random walks through the graph representation of the biological network and monitoring the interacting elements encountered during the random walks; and
using information about the random walks to define positions of interacting elements in the vector representation.

13. (canceled)

14. The method of claim 1, wherein converting the graph representation of the biological network to a vector representation comprises employing information about a hierarchy of the interacting elements of the biological network.

15. The method of claim 14, wherein converting the graph representation of the biological network to a vector representation comprises presenting the hierarchy of the interacting elements of the biological network in a hyperbolic space.

16. The method of claim 15, wherein presenting the hierarchy of the interacting elements of the biological network in a hyperbolic space comprises performing a hyperbolic embedding.

17. The method of claim 16, wherein the hyperbolic embedding is a Poincare embedding.

18-20. (canceled)

21. The method of claim 1, further comprising producing the engineered variant of the microorganism or the plurality of related microorganisms;
and testing the engineered variant of the microorganism or the plurality of related microorganisms.

22. (canceled)

23. An engineered variant microorganism comprising a modification to a gene or a gene-promoter combination of the microorganism or the plurality of related microorganisms, wherein the modification is selected using the method of claim 1.

24. (canceled)

25. A method of identifying promoter-gene combinations for editing in a microorganism genome, the method comprising:
(a) receiving a graph representation of a biological network of interacting elements of a microorganism or a plurality of related microorganisms, wherein the interacting elements comprise genes present in the microorganism or microorganisms;
(b) converting the graph representation of the biological network to a vector representation having locations of the interacting elements, which locations are derived from positions of said interacting elements in the graph representation;
(c) determining distance relationships between the interacting elements, including at least some of the genes present in the microorganism or microorganisms, as represented in the vector representation; and
(d) from the distance relationships determined in (c), ascribing probabilities of promoter-gene combinations for use in an engineered variant of the microorganism or the plurality of related microorganisms.

26. The method of claim 25, wherein ascribing probabilities of promoter-gene combinations comprises using the distance relationships to generate sampling probabilities for selecting a subset of the genes present in the microorganism or microorganisms.

27. The method of claim 25, wherein ascribing probabilities of promoter-gene combinations comprises using angles between interacting elements in the vector representation to suggest a first promoter from among a plurality of promoters for use with a first one of the genes present in the microorganism or microorganisms.

28. The method of claim 25, wherein using angles between interacting elements in the vector representation comprises determining cosine similarities between interacting elements in the vector representation.

29-32. (canceled)

33. The method of claim 25, wherein the graph representation of the biological network comprises two or more interacting elements selected from the group consisting of genes, compounds, reactions, genetic ontology terms, transcriptional units, and proteins.

34-35. (canceled)

36. The method of claim 25, wherein converting the graph representation of the biological network to a vector representation comprises:
conducting a plurality of random walks through the graph representation of the biological network and monitoring the interacting elements encountered during the random walks; and
using information about the random walks to define positions of interacting elements in the vector representation.

37. The method of claim 36, wherein the information about the random walks comprises frequencies at which the interacting elements of the graph representation were encountered during the random walks.

38. The method of claim 25, wherein converting the graph representation of the biological network to a vector representation comprises employing information about a hierarchy of the interacting elements of the biological network.

39. The method of claim 38, wherein converting the graph representation of the biological network to a vector representation comprises presenting the hierarchy of the interacting elements of the biological network in a hyperbolic space.

40. The method of claim 39, wherein presenting the hierarchy of the interacting elements of the biological network in a hyperbolic space comprises performing a hyperbolic embedding.

41. The method of claim 40, wherein the hyperbolic embedding is a Poincare embedding.

42-50. (canceled)

51. A system for creating a variant microorganism for producing a product, the system comprising:
(a) a computing device comprising one or more processors and memory, wherein the computing device is configured to:
(i) receive a graph representation of a biological network of interacting elements of a microorganism or a plurality of related microorganisms,
(ii) convert the graph representation of the biological network to a vector representation having locations of the interacting elements, which locations are derived from positions of said interacting elements in the graph representation,
(iii) determine distance relationships between the interacting elements as represented in the vector representation, and
(iv) from the distance relationships determined in (c), recommend a subset of the interacting elements for modification in an engineered variant of the microorganism or the plurality of related microorganisms; and
(b) a genetic engineering tool configured to produce the variant microorganism.

52. The system of claim 51, further comprising a bioreactor configured to produce the product from the modified organism.

53. The system of claim 51, wherein the genetic engineering tool configured to produce the modified organism is configured to apply a mutation to the gene or knock out the gene.

54. The system of claim 53, wherein the genetic engineering tool configured to produce the modified organism comprises a gene editing tool.

55. The system of claim 53, wherein the gene editing tool is a TALEN system, a zinc finger system, or a CRISPR/Cas9 system designed to apply the mutation to the gene.

56-70. (canceled)
